(19) United States
(12) Patent Application Publication
(10) Pub. No.: US 2026/0240733 A1
(43) Pub. Date: Aug. 20, 2026

(54) Microcapsules comprising a UV filter

(71) Applicant: KAPSERA, BORDEAUX (FR)

(72) Inventors: Wafa BOUHLEL, ANTIBES (FR); Edouard DULIEGE, MONTFORT SUR MEU (FR)

(21) Appl. No.: 18/879,037

(22) PCT Filed: Jun. 29, 2023
(86) PCT No.: PCT/EP2023/067857

§ 371 Date: Dec. 26, 2024

(30) Foreign Application Priority Data
Jun. 30, 2022 (FR) FR2206605

(51) Int. Cl. A61K 8/11; A23K 40/30; A23P 10/30; C02F 1/68

(52) U.S. Cl. CPC A61K 8/11; A23K 40/30; A23P 10/30; C02F 1/68

(57) ABSTRACT

A microcapsule with a core surrounded by a gelled shell, wherein the core further includes at least one active agent, and the shell includes dispersed colloidal UV filter particles, a method for producing the microcapsule, and its uses in various fields of application.

### CROSS-REFERENCE TO RELATED APPLICATIONS

This application claims benefit under 35 USC § 371 of PCT Application No. PCT/EP2023/067857 entitled MICROCAPSULES COMPRISING A UV FILTER, filed on Jun. 29, 2023 by inventors Wafa Bouhlel, Karen Chaitou and Edouard Duliege. PCT Application No. PCT/EP2023/067857 claims priority of French Patent Application No. 22 06605, filed on Jun. 30, 2022.

### FIELD OF THE INVENTION

The present invention relates to a microcapsule with an aqueous, oily or O/W and W/O emulsion liquid core, surrounded by a gelled shell, wherein the core further comprises at least one active agent, and the shell comprises dispersed colloidal UV filter particles, a method for producing said microcapsule, and uses thereof in various fields of application such as crop and/or seed treatment, human and/or animal nutrition, cosmetics and pharmaceuticals, and soil and wastewater decontamination.

### BACKGROUND OF THE INVENTION

Exposure of active compounds, particularly organic compounds, to ultraviolet (UV) light leads to the formation of free radicals through oxidation reactions. In organic molecules, the DNA bases that make up the genome undergo mutations that can be deleterious to microorganisms.

The energy emitted by the sun which reaches the Earth's surface can be divided as follows: 5% UV radiation, 45% visible radiation and 50% infrared radiation. Ultraviolet rays are the most destructive because of their higher energy content.

The invention relates to the use of a concentrated suspension of colloidal UV filter particles within the shell of a capsule to enable the active agent at the core of the capsule to be protected. In the case of microorganisms, the inventors have discovered that this incorporation improves their viability following exposure to ultra-violet radiation.

UV filters are molecules or particles that absorb, reflect and/or diffuse ultra-violet radiation. They are often used in cosmetics, in the form of nanoparticles, to protect against the harmful effects of ultraviolet rays on the skin.

The effectiveness of a UV filter depends on its ability to absorb, reflect, and/or diffuse light. Several physical/chemical parameters influence these physical phenomena, such as particle size, refractive index, and concentration.

### SUMMARY OF THE DESCRIPTION

Thus the invention relates to a microcapsule with a liquid core surrounded by a gelled shell wherein the microcapsule has an average diameter of between 50 and 4000 μm when in hydrated form, and wherein:
a. the core further comprises at least one active agent, and
b. the shell comprises dispersed colloidal UV filter particles, wherein the UV filter is not a soluble antioxidant compound.

The invention further relates to a method for producing microcapsules comprising the following steps:
a. separately conveying, in a double-layer conduit, a first liquid solution comprising at least one active agent and a second liquid solution comprising a biopolymer having gelling properties and dispersed colloidal UV filter particles;
b. forming a series of drops at the exit of the double-layer conduit, each drop comprising a central core formed from the first solution and a peripheral film formed from the second solution and completely covering the central core;
c. immersing each drop in a gelling solution capable of reacting with the biopolymer having gelling properties to make it change from a liquid state to a gelled state and form the gelled envelope, with the central core forming the liquid core;
d. recovering the formed capsules.

The invention further relates to the microcapsule obtainable by this method.

The purpose of the invention is to use microcapsules for any purpose aimed at protecting the active agent from the harmful effect of UV radiation. For example, when capsules are stored in extreme sunlight conditions, during a method that involves a step entailing exposure to UV light, or during use/application in various fields such as agriculture, food processing, cosmetics, or pharmaceuticals.

The object of the invention is the use of microcapsules for the treatment of plant crops and/or seeds thereof, for animal nutrition and/or feed, for human nutrition and/or feed, for the formulation of cosmetics and/or pharmaceutical compositions and for soil and wastewater decontamination.

The invention further relates to pharmaceutical compositions comprising a microcapsule according to the invention.

The invention further relates to a method for treating crops and/or their seeds comprising spreading, foliar spraying, or coating seeds with a suspension of microcapsules according to the invention.

### BRIEF DESCRIPTION OF THE DRAWINGS

FIG. 1: Evolution of the percentage viability of Bacillus sp. encapsulated with carbon black as a function of the time of exposure to artificial ultraviolet light.

FIG. 2: Survival of bacteria exposed to UV light. These curves represent the survival of Paraburkholderia phytofirmans PsJN bacteria in suspension (dotted curve) and the survival of bacteria encapsulated in microcapsules according to the invention using biochar as a UV filter.

FIG. 3: Comparison of the survival rate of bacteriophages encapsulated with biochar in the shell with that of bacteriophages encapsulated without biochar, and with that of suspended bacteriophages exposed to artificial ultraviolet radiation.

### DETAILED DESCRIPTION OF EMBODIMENTS

### Microcapsule

“Microcapsule” in this context means a capsule with an average diameter of less than 10 mm and comprising at least a core and a shell. Such capsules preferably comprise a liquid core encapsulated by a substantially solid gelled envelope. This type of capsule has applications in many technical fields. The shell comprises one or more concentric or non-concentric compartments. Preferably, the microcapsules according to the invention comprise a single core coated by the shell.

These microcapsules are therefore very different from microbeads, as microbeads are mainly made up of a solid or gelled matrix comprising multiple small inclusions.

The use of microcapsules rather than microbeads also makes it possible to confine microorganisms without immobilising them.

According to a preferred embodiment of the invention, the microcapsules have a core volume to total microcapsule volume ratio of greater than 20%. These microcapsules make it possible to protect a large core volume, and therefore active agent volume, for a given volume of shell.

Microcapsules are well known to the skilled person and can be formed by different techniques and have different shell compositions.

Typically, the microcapsules used in the context of the invention are produced according to the manufacturing method described in French patent no. 2939012.

As described below, the microcapsules according to the invention may be dehydrated. However, when they are in hydrated form, such as in suspension in an aqueous solution, the microcapsules according to the invention have an average diameter of between 50 and 4000 μm, preferably between 100 and 2000 μm, more particularly between 200 and 1000 μm, advantageously between 200 and 600 μm. This average diameter can be measured by various techniques well known to those skilled in the art, such as granulometry based on the diffraction of laser light, fractionation by sieving, or imaging by optical microscopy.

In one embodiment, the microcapsules according to the invention also comprise an intermediate layer between the core and the shell. Preferably this layer is composed of at least one biopolymer in solution or hydrogel form. Among other things, this makes it possible to limit any interactions between the UV filter and the active agent.

Preferably, the microcapsules according to the invention are free, i.e. they are not included in another structure such as a film, a bead, a gel, or encapsulated a second time, but they are in direct contact with the medium surrounding them, typically a liquid (if they are in suspension, for example) or a gas.

### The Core

The core of the microcapsules according to the invention is a liquid core comprising at least one active agent. Preferably, the viscosity of the core is less than 2000 mPa·s.

The core of the microcapsules according to the invention may be aqueous, oily or in the form of an oil-in-water (O/W) or water-in-oil (W/O) emulsion.

“Aqueous core” means a core based on a predominantly aqueous phase in which the active agent is dispersed or solubilised.

“Oily core” means a core based on a predominantly oily phase in which the active agent is dispersed or solubilised.

If the core is an O/W emulsion, the hydrophobic active agent will be dispersed in the oil droplets. This type of emulsion also makes it possible to encapsulate both a hydrophobic active agent and a hydrophilic active agent.

“Active agent” means an active substance, active principle, or active ingredient that is known and/or used for a particular purpose. Preferably the active agent is sensitive to UV, i.e. exposure to UV can cause irreversible changes to the active agent, potentially reducing its activity.

Advantageously, the active agent is chosen from microorganisms, natural extracts, semiochemical compounds and mixtures thereof.

“Microorganisms” means living organisms that are invisible to the naked eye and can only be observed through a microscope, in particular bacteria, fungi, microalgae and viruses.

“Natural extracts” means extracts of animal, vegetable or mineral origin and, for example, extracts of plants, algae, minerals, and microorganisms, such as essential oils, etc.

For example, in one embodiment, the active agent is a biocontrol agent, a biofertiliser, and/or a biostimulant.

“Biocontrol agent” means an agent designed to protect crops and/or their seeds against diseases or pests, based on the use of natural mechanisms. These agents comprise macro-organisms (insects and nematodes), phytopharmaceutical products composed of microorganisms (viruses, bacteria and fungi), chemical mediators (pheromones and kairomones), and natural substances of plant, animal or mineral origin.

“Biofertiliser” means a non-pathogenic substance or living microorganism capable of increasing the availability of nutrients to plants.

“Biostimulant” means a non-pathogenic substance or living microorganism capable of stimulating natural processes to improve/advance nutrient uptake, nutrient efficiency, tolerance to abiotic stresses, and/or crop quality, irrespective of the nutrient content of the biostimulant.

In another embodiment, the active agent may be a macronutrient (carbohydrates, lipids and proteins) or a micro-nutrient (vitamins, minerals, trace elements or amino acids), preferably intended to provide organoleptic or nutritional properties. In this way, the active agent can be a food supplement, an agent that facilitates digestion, and/or the absorption of certain compounds. For example, the active agent in the microcapsule according to the invention may be: a vitamin, an amino acid or a fatty acid.

In another embodiment, the active agent may be a cosmetic product. Cosmetic product means any substance or mixture intended to be placed in contact with the exposed parts of the human body (epidermis, hair and follicular system, nails, lips, and external genitalia) or with the teeth and oral mucous membranes with a view exclusively or primarily to cleaning them, perfuming them, modifying their appearance, protecting them, keeping them in good condition, or correcting body odours.

In another embodiment, the agent may be an active principle used in the composition of a medication and which has a therapeutic or preventive effect.

In another embodiment, the agent can be an active principle used for removing pollution from water or soil, for example in water treatment plants.

Preferably, the core of the capsule according to the invention does not comprise UV filter particles.

### The Shell

Microcapsules according to the invention preferably comprise at least one liquid core encapsulated by a substantially solid gelled envelope called the shell.

Preferably, the shell of the microcapsules according to the invention is mainly composed of a biopolymer with gelling properties; this biopolymer that forms a majority of the shell is hereinafter referred to as the main biopolymer. Examples of such biopolymers with gelling properties are alginate, gellan gum, xanthan gum, pectin, chitosan, agar, and carrageenan.

The materials that make up the shell are preferably biodegradable and biosourced. The shell is preferably semi-permeable to gases and low-molecular-weight molecules.

The gels forming the shell can be physical or chemical, i.e. formed by coacervation or polymerisation.

The gelling of these biopolymers can be achieved by a variation in temperature (gellan gum), a variation in pH (chitosan, pectin) or by ions (alginate, carrageenan).

Preferably, the shell of the microcapsules according to the invention is mainly composed of a biopolymer having gelling properties caused by ionic or temperature variation.

Preferably, the shell of the microcapsules according to the invention is mainly composed of alginate.

The shell may also comprise one or more biopolymers other than the main biopolymer, such as starch (in its various forms, e.g. amylose, pregelatinised starch), potato protein, or a biopolymer other than the main biopolymer having gelling properties, e.g. alginate, gellan gum, xanthan gum, pectin, chitosan, agar, or carrageenan.

Preferably, the shell of the microcapsules according to the invention comprises a gel containing water, one or more biopolymers with gelling properties, and optionally a surfactant resulting from its manufacturing method.

Preferably, the shell of the microcapsules according to the invention comprises a gel containing water, alkali alginate, and optionally a surfactant resulting from its manufacturing method.

Preferably the alkali alginate is a sodium alginate or a potassium alginate. Alginates are produced from brown algae that are also called seaweed. Such alginates advantageously have an α-L-guluronate content of over 50%, preferably over 55% or even over 60%.

The surfactant is advantageously an anionic surfactant, a non-ionic surfactant, a cationic surfactant or a mixture thereof. The molecular weight of the surfactant is between 150 g/mol and 10,000 g/mol, advantageously between 250 g/mol and 1,500 g/mol.

If the surfactant is an anionic surfactant, it is chosen, for example, from an alkyl sulphate, an alkyl sulphonate, an alkyl aryl sulphonate, an alkaline alkyl phosphate, a dialkyl sulphosuccinate, and an alkaline earth salt of saturated or unsaturated fatty acids. These surfactants advantageously have at least one hydrophobic hydrocarbon chain with a carbon number greater than 5, or even 10, and at least one hydrophilic anionic group, such as a sulphate, sulphonate, or carboxylate linked to one end of the hydrophobic chain. If the surfactant is a cationic surfactant, it is for example chosen from an alkylpyridium or alkylammonium halide salt such as n-ethyldodecylammonium chloride or bromide, cetylammonium chloride or bromide (CTAB). These surfactants advantageously have at least one hydrophobic hydrocarbon chain with a carbon number greater than 5, or even 10, and at least one hydrophilic cationic group, such as a quaternary ammonium cation. If the surfactant is a non-ionic surfactant, it is chosen, for example, from polyoxyethylenated and/or polyoxypropylenated derivatives of fatty alcohols, fatty acids, or alkylphenols, arylphenols, or from alkyl glucosides, polysorbates and cocamides.

In one embodiment, the surfactant is sodium lauryl sulphate (SLS) also known as sodium dodecyl sulphate and/or polyoxyethylene sorbitan monoleate (Polysorbate 80).

Preferably, the surfactant is polyoxyethylene sorbitan monoleate (Polysorbate 80).

In one embodiment, the mass content of surfactant in the shell is greater than 0.001% and advantageously greater than 0.1%. Advantageously, the concentration by mass of surfactant is approximately 0.03%.

The shell of the microcapsule preferably has a thickness of between 0.1% and 30%, advantageously between 1% and 20%, and more particularly between 10% and 20% of the diameter of the capsule.

The shell of the microcapsule further comprises dispersed colloidal UV filter particles. Preferably, the UV filter particles are in the shell of the microcapsule, and advantageously are not in the core of the microcapsule. This makes it possible to use less filter for equivalent UV protection, but also to limit interactions between the UV filter and the active agent.

“Colloidal UV filter particles” means particles with an average size of between 10 nm and 10 μm and more preferably between 100 nm and 5 μm. In general, the smaller the particles, the more effective they are against UV rays. These filters can also be used to opacify the microcapsules.

Preferably, the UV filter is not a soluble antioxidant compound. These compounds tend to degrade more rapidly when exposed to UV light.

The UV filter according to the invention may be organic or inorganic, or a mixture of organic and inorganic filters.

Preferably, the UV filter is chosen from titanium oxide, carbon black, biochar, charcoal, latex, silica, clays, and mixtures thereof.

Biochar is the product of biomass pyrolysis and is used as an agricultural soil amendment.

Examples of clays used as UV filters are talc or kaolin and mixtures thereof.

Kaolin is a type of clay used to filter ultraviolet rays. It is also used to protect certain crops from agricultural pests.

Preferably the UV filter according to the invention is biodegradable and/or edible and/or cosmetically acceptable and/or pharmaceutically acceptable.

“Cosmetically acceptable” in this context refers to compositions and molecular entities that do not produce side effects, allergic reactions, or otherwise undesirable reactions when administered to a subject. A cosmetically acceptable UV filter is therefore compatible with the skin, integuments and/or mucous membranes, and does not induce sensations of discomfort or, more generally, disorders likely to lead the user to suspend or stop administration of the cosmetic composition comprising it.

“Pharmaceutically acceptable” in this context refers to compositions and molecular entities that do not produce side effects, allergic reactions, or otherwise undesirable reactions when administered to a subject. Thus a pharmaceutically acceptable UV filter does not induce disorders likely to lead the user to suspend or stop administration of the pharmaceutical composition comprising it.

“Dispersed UV filter particles” refers to particles dispersed in the shell of the microcapsule, in order to achieve a uniform UV protection effect over the surface of the microcapsule.

The microcapsule according to the invention may be in dehydrated form. Preferably the dehydration is partial, and the microcapsules according to the invention have a moisture content of less than 10%, measured by a moisture analyser after dehydration.

In dehydrated form, the average diameter of the microcapsule tends to decrease. Preferably, the microcapsule according to the invention has an average diameter of between 10 μm and 4 mm in dehydrated form.

### Production Method

The invention further relates to a method for producing microcapsules comprising the following steps:
a. separately conveying, in a double-layer conduit, a first liquid solution comprising at least one active agent and a second liquid solution containing a biopolymer having gelling properties and dispersed colloidal UV filter particles;
b. forming a series of drops at the exit of the double-layer conduit, each drop comprising a central core formed from the first solution and a peripheral film formed from the second solution and completely covering the central core;
c. immersing each drop in a gelling solution capable of reacting with the biopolymer having gelling properties to make it change from a liquid state to a gelled state and form the gelled envelope, with the central core forming the liquid core;
d. recovering the formed capsules.

Preferably, the invention relates to a method for producing microcapsules comprising the following steps:
a. separately conveying, in a double-layer conduit, a first liquid solution comprising at least one active agent and a second liquid solution containing a biopolymer containing an alkali alginate and dispersed colloidal UV filter particles;
b. forming a series of drops at the exit of the double-layer conduit, each drop comprising a central core formed from the first solution and a peripheral film formed from the second solution and completely covering the central core;
c. immersing each drop in a gelling solution containing a reagent capable of reacting with the alkali alginate to convert it from a liquid state to a gelled state and form the gelled envelope, with the central core forming the liquid core;
d. recovering the formed capsules.

The definitions laid out in the microcapsule section also apply here.

In one embodiment of the methods according to the invention, the second solution contains at least one surfactant before it comes into contact with the first solution.

The surfactant is preferably chosen from an anionic surfactant, a cationic surfactant, a non-ionic surfactant, or mixtures thereof.

For example, said surfactant is an alkyl sulphate, an alkyl sulphonate, an alkylaryl sulphonate, an alkaline alkyl phosphate, a dialkyl sulphosuccinate, an alkaline-earth salt of saturated or unsaturated fatty acids, an alkylpyridium or alkylammonium halide salt such as nethyldodecylammonium chloride or bromide, cetylamonium chloride or bromide, polyoxyethylenated and/or polyoxypropylenated derivatives of fatty alcohols of fatty alcohols, fatty acids or alkylphenols, or arylphenols, alkyl glucosides, polysorbates, cocamides, or mixtures thereof.

In one embodiment, the surfactant is sodium lauryl sulphate (SLS) also known as sodium dodecyl sulphate and/or polyoxyethylene sorbitan monoleate (Polysorbate 80).

Preferably, the surfactant is polyoxyethylene sorbitan monoleate (Polysorbate 80).

The total percentage by mass of surfactant in the second solution is preferably greater than 0.01% and is advantageously between 0.01% and 0.5% by mass.

The mass content of biopolymer with gelling properties in the second solution is advantageously less than 5% by mass and is preferably between 0.5 and 3% by mass.

Preferably, in the case of the production of a microcapsule with a shell predominantly comprising alginate, the mass content of alkali alginate in the second solution is advantageously less than 5% by mass and is preferably between 0.5 and 3% by mass.

Preferably, the alkali alginate in the second solution has an α-L-guluronate block content of more than 50%, in particular more than 55%.

Preferably, the second liquid solution comprises 0.1 to 20% v/v UV filter.

The ratio of the flow rate of the first solution to the flow rate of the second solution at the outlet of the double-layer conduit is between 0.01 and 100, advantageously between 0.05 and 50 and more particularly between 0.1 and 10, the gelled envelope having a thickness of between 0.1% and 30%, advantageously between 1% and 20%, and particularly between 10% and 20% of the diameter of the capsule, after recovery of the capsules formed.

“Gelling solution suitable for reacting with the biopolymer” means a solution at a particular temperature, or a particular pH, or comprising a particular reagent enabling the biopolymer in the second liquid solution to change from a liquid state to a gelled state.

In the case of the production of a microcapsule with a shell predominantly comprising alginate, the gelling solution is for example an aqueous solution of a reagent of the XₙIₘ type, where X is advantageously a halide ion such as a chloride ion, a bromide ion, an iodide ion or a fluoride ion, and I is advantageously a multivalent cation of an alkaline-earth metal such as calcium, magnesium or barium, and n and m are greater than or equal to 1. Preferably, the gelling solution further comprises a surfactant such as polysorbate 20 or polysorbate 80.

The multivalent ions present in the gelling solution thus formed are capable of reacting with the alginate to form bonds between the different alginate chains present in the second solution, when the second solution comes into contact with the gelling solution. In the case where the alginate is a sodium alginate (NaAlg), and the reagent is calcium chloride, the reaction that occurs is as follows:

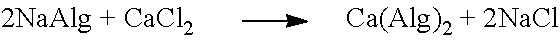

In methods according to the invention, the concentration of reagent in the gelling solution is advantageously between 5% and 20% by mass.

Preferably, the gelling solution is placed below and away from the outlet of the double-layer conduit, so that the drops formed by coextrusion in the double-layer conduit fall spontaneously by gravity through a volume of air into the gelling solution in which they are immersed.

In one embodiment, the microcapsules formed may follow an additional rinsing step by passing through a rinsing and storage solution consisting essentially of water.

The method according to the invention may further comprise a step (step e.) of dehydrating the microcapsules. This step can be carried out using techniques well known to the person skilled in the art, for example hot drying techniques or cold drying techniques. This step is preferably carried out by steaming, freeze-drying, or osmotic dehydration. In one particular example, dehydration is carried out by drying in a fluidised air bed and/or by freeze-drying. Preferably, the dehydration step removes at least 90% of the water (by weight) from the microcapsules.

The invention further relates to microcapsules that can be obtained by the method according to the invention.

### Uses and Methods

The definitions laid out in the microcapsule section also apply here.

Preferably, in the methods and uses according to the invention, the microcapsules are used as they are, i.e. they are not included in another structure (such as a film, a bead, a gel, or encapsulated a second time), but rather are used directly, possibly suspended in a liquid.

The invention concerns the use of microcapsules according to the invention for the treatment of plant crops and/or their seeds.

The term “treatment of plant crops and/or their seeds” refers to biocontrol, biofertilisation and/or biostimulation treatments that may be applied before, during or after planting.

In fact, the active agent in the microcapsules according to the invention may be an agent with effects on plant crops and/or their seeds. In particular, the active agent can be used to protect crops and/or their seeds against diseases or pests, but also to act as a fertiliser and/or to promote the growth of plant crops and/or their seeds.

The invention therefore relates in particular to the use of microcapsules for treating plant crops and/or their seeds, wherein the active agent is a biocontrol agent, a biofertiliser, and/or a biostimulant. The microcapsules can be used suspended in a liquid or in dehydrated solid form.

The invention comprises a method of treating crops and/or seeds comprising the application of microcapsules according to the invention. The invention therefore further relates to a method for treating crops and/or seeds comprising spreading, foliar spraying or coating seeds with microcapsules according to the invention, preferably microcapsules according to the invention wherein the active agent is a biocontrol agent, a biofertiliser and/or a biostimulant. In this method, the microcapsules can be used suspended in a liquid or in dehydrated solid form.

In another embodiment, the invention relates to the use, preferably non-therapeutic, of microcapsules according to the invention for animal and/or human nutrition and/or diets. In this embodiment, the active agents used are preferably not therapeutic active agents, i.e. they are of interest from a nutritional or dietary point of view but they do not make it possible to prevent or treat illnesses in the subject consuming them. Preferably, the active agents used are edible agents, preferably with organoleptic or nutritional properties. In this application, the active agents are preferably of interest from a nutritional or dietary point of view, but do not prevent or treat diseases in the subject consuming them.

“Diet” in this context means the habitual or frequent intake of food. Microcapsules can contain a variety of active agents to improve the properties of a food product, such as its organoleptic properties, shelf life or bioavailability.

“Nutrition” in this context means taking food supplements, often on an occasional basis or as part of a cure, typically to prevent or make up for a deficiency. Microcapsules according to the invention may contain a variety of active agents to improve nutrient intake by improving their bioavailability or to modify intestinal flora.

“Animal” in this context means a wild or domesticated animal. It is preferably a domesticated, farmed or pet animal. In particular, the animals according to the invention are chosen from pets such as dogs, cats, fish, rabbits, horses, tortoises, farmed species such as cattle (cows, oxen), sheep, goats, rabbits, pigs (pig), camels, and birds raised in poultry farming (hens, quails, etc.).

The invention further relates to the use of microcapsules according to the invention for the formulation of cosmetics. In this embodiment, the active agents used are preferably not therapeutic active agents, i.e. they are of interest from a cosmetic point of view but they do not make it possible to prevent or treat illnesses in the subject using them.

The invention further relates to the use of microcapsules according to the invention for the formulation of pharmaceutical compositions. In this embodiment, the active agents used are preferably therapeutic active agents, i.e. they make it possible to prevent or treat illnesses in the subject consuming them.

The use of microcapsules in accordance with the invention makes it easier to store, package or prepare cosmetic or pharmaceutical formulations, while keeping the active agent in good quality.

### Pharmaceutical Compositions and Therapeutic Applications

The invention further relates to a pharmaceutical composition comprising a microcapsule according to the invention. The application further relates to a microcapsule according to the invention for use as a medication.

The present invention further relates to a method of treating a subject, comprising administering a therapeutically effective amount of microcapsules according to the invention to a subject in need thereof.

In this embodiment, the microcapsule according to the invention advantageously comprises a therapeutic active agent.

“Therapeutically active agent” in this context means an active agent as defined in the microcapsule section above which in addition is known and/or used for a particular therapeutic purpose, i.e. it is known and/or used in the treatment or prevention of diseases.

The term “pharmaceutical composition” as defined herein refers to a mixture or solution comprising at least one therapeutic agent to be administered to a subject in order to prevent or treat a particular disease affecting the subject.

Pharmaceutical compositions as defined herein therefore preferably also comprise pharmaceutically acceptable excipients.

“Pharmaceutically acceptable” in this context refers to compositions and molecular entities that do not produce side effects, allergic reactions, or otherwise undesirable reactions when administered to a subject.

“Subject” in this context means a living being, preferably a mammal, and more particularly a human being.

“Therapeutically effective quantity” in this context means a quantity that is effective at the doses and for the periods required to obtain the desired therapeutic result. This amount may vary depending on factors such as the disease, the extent of the disease, the age, sex and weight of the subject, and the ability of the microcapsules to produce a desired therapeutic result. A therapeutically effective quantity is one in which any toxic or harmful effects are offset by therapeutically beneficial effects. A therapeutically effective quantity also encompasses a quantity sufficient to confer a benefit, for example a clinical benefit.

Such pharmaceutical compositions are preferably adapted to the route of administration.

In a particular embodiment, the pharmaceutical compositions are suitable for oral, sublingual, buccal, intranasal, or topical administration.

The invention further relates to the use of microcapsules according to the invention for manufacturing a medication. The invention further relates to the use of microcapsules according to the invention for the formulation of pharmaceutical compositions.

For these uses, the microcapsule according to the invention advantageously comprises a therapeutic active agent.

This is because the use of microcapsules according to the invention makes it easier to store, package, or prepare pharmaceutical compositions while preserving the active agents.

“Treatment” or “treating” in this context means partially or substantially achieving one or more of the following results: Partially or totally reducing the extent of the disease; improving a clinical symptom or an indicator associated with the disease; delaying, inhibiting or preventing the progression of the disease.

“Prevention” or “preventing” in this context means partially or substantially achieving one or more of the following results: Preventing or delaying the onset of the disease or at least one of its symptoms, preventing or delaying the deterioration of an indicator associated with the onset of the disease.

The invention further relates to the use of microcapsules according to the invention for soil or wastewater decontamination. Such compositions make it possible to protect the active agent used for decontamination from UV rays, for example when it is used in a water treatment plant or on the surface of the ground.

In the description and in the following examples, unless otherwise indicated, percentages are percentages by weight and ranges of values are expressed as “between . . . and . . . ”, “from . . . to . . . ” or “greater than . . . ” include the specified limits.

Throughout the application, the wording “comprising one” or “including one” means “comprising at least one” or “including at least one” unless otherwise specified.

The following examples are given by way of illustration and are not intended to limit the scope of the invention.

### EXAMPLES

### Example 1

Laboratory tests on bacterial samples were subjected to an accelerated ageing test. A comparison was made of the survival of Bacillus sp. bacteria in suspension (non-encapsulated) and encapsulated, when exposed to artificial ultraviolet light. The bacteria were encapsulated in a shell made of alginate and carbon black, which is a known ultraviolet filter. The source of the UV rays used is an oven containing a mercury vapour lamp whose power is around 18 times greater than that of solar radiation (on a summer's day at midday in the United States). This means that approximately one minute in the oven is equivalent to 18 minutes in the field. Two exposure times were tested: Three minutes and five minutes.

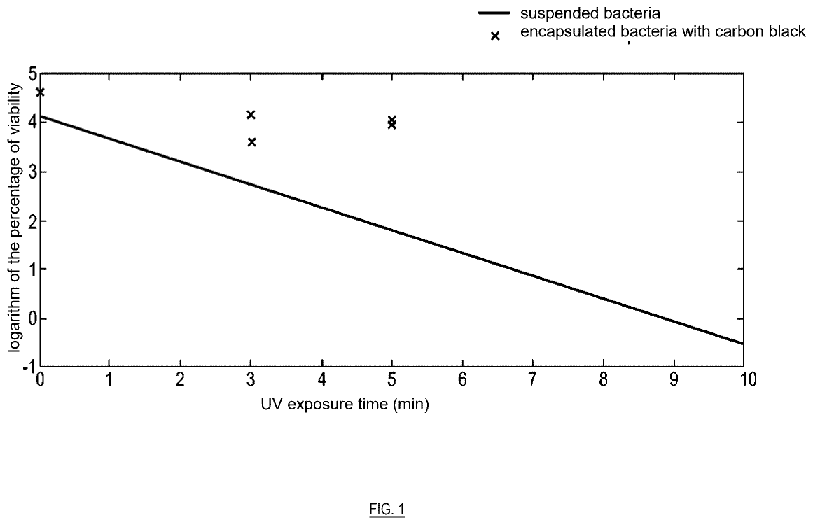

The results are shown in FIG. 1. The inventors have observed that the percentage of viability is greater for encapsulated bacteria than for those in suspension. After five minutes' exposure, equivalent to one-and-a-half hours in the field, the survival of bacteria in suspension was 1%, while the survival of encapsulated bacteria was 57%.

### Example 2

### Encapsulation Operating Conditions:

Core solution (or core fluid): bacterial suspension in a TSB medium (tryptone soy broth) diluted 1:10 to limit bacterial proliferation during UV exposure.

Shell solution (or shell fluid): 1.8 wt. % alginate solution, 0.5 mM SDS surfactant, 1.5% v/v biochar.

The flow ratio between core and shell suspension is set at 0.5. For this reason, the inventors want to maximise the thickness of the hull.

Total fluid flow is 340 mL/h.

Under these conditions, the capsules formed had an average diameter of 516 μm with a coefficient of variation (CV) of 17%.

Two UV filters were selected for testing: Biochar and kaolin.

These two candidates were characterised to check their compatibility with the microencapsulation method. The distribution of particle sizes was studied. In addition, the absorbance of ultraviolet rays by these compounds was studied spectrophotometrically. The inventors have observed that for the same volume fraction (0.01%), the absorbency of ultraviolet rays by kaolin is slightly higher than that of biochar.

The effectiveness of UV protection with biochar was evaluated by determining the survival rate of bacteria encapsulated with biochar in the shell compared with that of bacteria in suspension, and with that of bacteria encapsulated without biochar, exposed to artificial UV radiation. The bacteria used for these tests belong to the Paraburkholderia phytofirmans species of the PsJN strain. These bacteria stimulate plant growth and help protect plants from certain biotic and abiotic stresses.

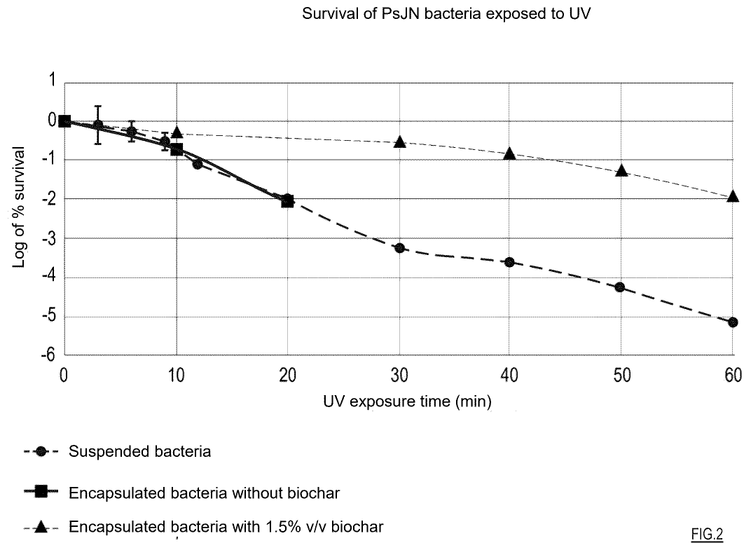

The results obtained from these tests are shown in FIG. 2. The survival rate of bacteria encapsulated with 1.5% v/v biochar was greater than that of bacteria in suspension or bacteria encapsulated without biochar. The survival rate of bacteria in suspension after 10 minutes' exposure was 8%, compared with 46% for bacteria encapsulated with biochar.

Encapsulation with biochar in the shell therefore represents a very interesting solution for protecting microorganisms from ultraviolet rays.

The performance of biochar in protecting against ultra-violet radiation was also assessed on bacteriophages of the M13K07 strain. Two prototype capsules were produced, and their performance assessed. Prototypes 1 and 2 correspond to wet alginate capsules without biochar and with biochar respectively.

The operating conditions for encapsulation are as follows:

The injector nozzle has a diameter of 200 μm. The total flow rate of core and shell fluid named Qtot is 500 mL/h, the flow ratio between core and shell fluid named Rq is 0.8 and confers a membrane thickness of at least 30 μm.

Core solution (or core fluid): bacteriophaage suspension in a physiological medium diluted 1:10 (to limit bacterial proliferation during UV exposure).

Shell solution (or shell fluid): 1.8 wt. % alginate solution, 0.5 mM SDS surfactant, 2.9% v/v biochar.

Under these conditions, the capsules of prototype 1 had an average diameter of 462 μm with a coefficient of variation (CV) of 17%.

The capsules of prototype 2 had an average diameter of 542 μm with a coefficient of variation (CV) of 18%.

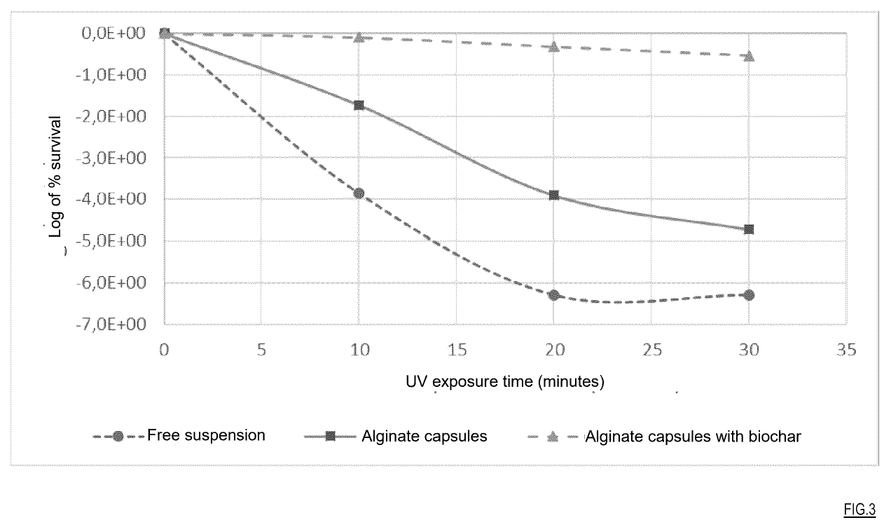

The survival rate of bacteriophages encapsulated with biochar in the shell, compared with that of bacteriophages encapsulated without biochar, and with that of suspended bacteriophages exposed to artificial ultraviolet radiation, was estimated. The results of these tests are shown in FIG. 3 and Table 1 below.

TABLE 1

| | survival | | log of % |
|---|---|---|---|
| exposure time | rate | % survival | survival |
| Free suspension | | | |
| 0 | 9.38E+08 | 100.00000% | 0.0E+00 |
| 10 | 1.28E+05 | 0.01368% | −3.9E+00 |
| 20 | 4.67E+02 | 0.00005% | −6.3E+00 |
| 30 | 4.67E+02 | 0.00005% | −6.3E+00 |
| Alginate capsules | | | |
| 0 | 2.82E+08 | 100.000% | 0.00E+00 |
| 10 | 5.30E+06 | 1.881% | −1.73E+00 |
| 20 | 3.53E+04 | 0.013% | −3.90E+00 |
| 30 | 5.26E+03 | 0.002% | −4.73E+00 |
| Alginate capsules | | | |
| with biochar | | | |
| 0 | 1.10E+08 | 100.0% | 0.0E+00 |
| 10 | 8.87E+07 | 80.3% | −9.5E−02: |
| 20 | 5.29E+07 | 47.9% | −3.2E−01 |
| 30 | 3.22E+07 | 29.2% | −5.3E−01 |

The survival rate of suspended bacteriophages after 30 minutes was 0.00005% (an approximate loss of 6 logs) compared with 30% (a loss of 0.5 logs) for bacteriophages in capsule form in the presence of biochar in the shell and a survival rate of 0.002% (an approximate loss of 4 logs).

These results confirm that microorganisms, in this case bacteriophages, are effectively protected against ultraviolet rays when they are in capsule form in the presence of biochar in the shell. In addition, a slightly higher survival rate was observed when the bacteriophages were encapsulated than when they were in suspension.

### Example 3

### Encapsulation Operating Conditions:

Core solution (or core fluid): inverse emulsion (W/O—water-in-oil) containing phenylalanine solubilised in the aqueous phase. This emulsion is prepared as follows: The aqueous phase is prepared by solubilising phenylalanine (960 mg) in 120 mL ultrapure water under stirring with a bar magnet for 30 minutes. 1.37 g of Polyglycerol Polyricinoleate (PGPR 90) surfactant was added to MCT oil. PGPR 90 is dispersed in the oil phase using a rotor-stator at 12,000 rpm for 2 minutes. The emulsion is then formed by introducing the aqueous phase into the oily phase while the rotor-stator is stirring. The rotor-stator is operated at 18,000 rpm during the introduction of the aqueous phase into the oil phase.

Shell solution (or shell fluid): 1.8 wt. % alginate solution, 0.5 mM SDS surfactant, 4 wt. % E-153 edible charcoal.

The flow ratio between the core emulsion and the shell solution is set at 0.5.

Total fluid flow is 350 mL/h.

The injector nozzle has a diameter of 145 μm.

UV exposure is achieved using a lamp emitting at 254 nm with an irradiance of 0.76 mW/cm². The samples were placed 10 cm from the source. The effectiveness of UV protection with activated carbon was assessed spectrophotometrically by measuring the absorbance at 280 nm of the aqueous phase of the emulsion at the exposure times t0, t20 min and t1 h for the emulsion alone, the encapsulated emulsion without edible charcoal and the emulsion encapsulated with edible charcoal.

The aqueous phase of the emulsion is extracted by introducing between 500 mg and 1 g of sample into 8 mL of PBS with 25 mM EDTA in a 15 mL tube placed for 20 minutes on a shaker. The tubes were then placed in an ultrasound bath for 8 minutes. Finally, the tubes were centrifuged at 4700 rpm for 15 minutes. The aqueous phase is then collected in each tube below the oily phase and filtered using a 0.2 μm pore size filter.

### Result

Phenylalanine exposed to this UV source is modified, and its absorbance measured at 280 nm increases as the exposure progresses. The lower the increase in absorbance, the more effective the UV protection.

The absorbance at 280 nm of the emulsion sample alone increased more than 300-fold after one hour. In the case of the encapsulated emulsion without edible charcoal, this absorbance was raised 13-fold, whereas for the capsules containing edible charcoal, it was limited to being raised less than two-fold.

TABLE 2
Absorbance at 280 nm of the aqueous phase of the
emulsion at exposure times t0, t20 min and t1 h

| UV exposure | | Encapsulated | Encapsulated |
|---|---|---|---|
| time | | emulsion without | emulsion with |
| (min) | Emulsion | edible charcoal | edible charcoal |
| 0 | 0.000174213 | 0.001090125 | 0.009997412 |
| 20 | 0.0164797 | 0.006557208 | 0.011018768 |
| 60 | 0.058285952 | 0.01441572 | 0.019759798 |

TABLE 3
Increase in absorbance at 280 nm of the aqueous phase of the
emulsion after exposure to UV light for 20 min and 1 h

| | | Encapsulated | Encapsulated |
|---|---|---|---|
| | | emulsion | emulsion |
| | | without edible | with edible |
| | Emulsion | charcoal | charcoal |
| at 20 min | 9360% | 502% | 10% |
| at 1 hour | 33357% | 1222% | 98% |

1. A microcapsule comprising a liquid core comprising:
a gelled shell surrounding the core, wherein the microcapsule has an average diameter of between 50 and 4000 μm when in hydrated form, the shell comprising a UV filter comprising dispersed colloidal UV filter particles, wherein the UV filter is not a soluble antioxidant compound; and
at least one active agent.

2. The microcapsule according claim 1, wherein said core does not comprise UV filter particles.

3. The microcapsule according to claim 1 comprising an intermediate layer between said core and said shell.

4. The microcapsule according to claim 1 in which said UV filter is organic or inorganic.

5. The microcapsule according to claim 1 in which said UV filter is chosen from titanium oxide, carbon black, biochar, charcoal, latex, silica, clays, and mixtures thereof.

6. The microcapsule according to claim 1 in which said at least one active agent is chosen from a microorganism, a natural extract, a semiochemical and mixtures thereof.

7. The microcapsule according to claim 1 in dehydrated form.

8. A method for producing microcapsules, comprising:
separately conveying, in a double-layer conduit, a first liquid solution comprising at least one active agent and a second liquid solution comprising a biopolymer having gelling properties and dispersed colloidal UV filter particles;
forming a series of drops at the exit of the double-layer conduit, each drop comprising a central core formed from the first solution and a peripheral film formed from the second solution and completely covering the central core;
immersing each drop in a gelling solution capable of reacting with the biopolymer having gelling properties to make it change from a liquid state to a gelled state and form the gelled envelope, with the central core forming the liquid core; and
recovering the formed capsules.

9. The method for producing microcapsules according to claim 8, wherein the second liquid solution comprises from 0.1 to 20% v/v UV filter.

10. The method of producing microcapsules according to claim 8 comprising dehydrating the microcapsules.

11. A microcapsule obtainable by the method according to claim 8.

12. A use of microcapsules according to claim 1 for the treatment of plant crops and/or their seeds.

13. A method for treating crops and/or seeds comprising spreading, foliar spraying, or coating seeds with a suspension of microcapsules according to claim 1.

14. The use of microcapsules according to claim 1 for animal nutrition and/or diets.

15. The use of microcapsules according to claim 1 for human nutrition and/or diets.

16. The use of microcapsules according to claim 1 for formulation of cosmetics.

17. The use of microcapsules according to claim 1 for formulation of pharmaceutical compositions.

18. A pharmaceutical composition comprising a microcapsule according to claim 1.

19. The use of microcapsules according to claim 1 for soil or wastewater decontamination.
